(19) United States
(12) Patent Application Publication
(10) Pub. No.: US 2019/0117649 A1
(43) Pub. Date: Apr. 25, 2019

(54) COMBINATION THERAPY FOR THE TREATMENT OF ACUTE MYELOID LEUKEMIA

(71) Applicant: ASTELLAS PHARMA INC., Chuo-ku (JP)

(72) Inventors: Erkut BAHCECI, Northbrook (IL); Yoko YAMAKI, Tokyo (JP)

(73) Assignee: ASTELLAS PHARMA INC., Chuo-ku (JP)

(21) Appl. No.: 16/089,603

(22) PCT Filed: Mar. 27, 2017
(86) PCT No.: PCT/JP2017/012293

§ 371 Date: Sep. 28, 2018

Related U.S. Application Data
(60) Provisional application No. 62/314,700, filed on Mar. 29, 2016.
(60) Provisional application No. 62/368,343, filed on Jul. 29, 2016.

(51) Int. Cl. A61K 31/497; A61K 31/706; A61P 35/02

(52) U.S. Cl. CPC A61K 31/497; A61P 35/02; A61K 31/706

(57) ABSTRACT

Provided are methods, uses, and compositions for treating acute myeloid leukemia which includes therapeutically effective combinations of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and 4-amino-1-β-D-ribofuranosyl-1,3,5-triazin-2(1H)-one, or a salt thereof.

### CROSS-REFERENCE TO RELATED APPLICATIONS

This application claims priority to U.S. Provisional Patent Application No. 62/314,700, filed 29 Mar. 2016, and Application No. 62/368,343, filed 29 Jul. 2016, the contents of which are incorporated by reference in its entirety.

### TECHNICAL FIELD

The present invention relates to methods, uses, and compositions for treating acute myeloid leukemia which includes therapeutically effective combinations of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and 4-amino-1-β-D-ribofuranosyl-1,3,5-triazin-2(1H)-one, or a salt thereof.

### BACKGROUND ART

Over 90% of leukemia cases are diagnosed in adults 20 years of age and older, among whom the most common types are chronic lymphocytic leukemia (35%) and acute myeloid (myelocytic) leukemia (AML) (32%) (Cancer Facts & Figures, Atlanta, American Cancer Society; 2014). The median age at diagnosis is 67 years of age, with 54% of patients diagnosed at 65 years or older (O′Donnell et al., Acute Myeloid Leukemia, J Natl Compr Cancer Network, 2012; 10:984-1021). It was estimated that 18,860 people (11,530 men and 7,330 women) were to be diagnosed with AML, and 10,460 were to die from the disease, in 2014 in the United States (Id., American Cancer Society, 2014). While 60% to 80% of younger patients achieve a complete remission (CR) with standard therapy, only about 30% to 40% of the overall patient population has long-term disease-free survival (Tallman, New strategies for the treatment of acute myeloid leukemia including antibodies and other novel agents, Hematology Am Soc Hematol Educ Program. 2005:143-50). Outcomes are worse for patients aged 60 years or over, with CR rates in the range of 40% to 55% and poor long-term survival rates.

FLT3 (FMS-like tyrosine kinase 3) is one of the most frequently mutated genes in AML. Activating mutations in FLT3 such as internal tandem duplications (ITD) at the juxtamembrane domain are present in approximately 25-30% of newly diagnosed AML cases. Patients with AML harboring the FLT3-ITD mutation have a poor prognosis following the current induction chemotherapy treatment of cytarabine (AraC) and an anthracycline (daunorubicin [DNR] or idarubicin [IDR]). Azacitidine is a treatment option for AML patients who are not eligible for intensive chemotherapy.

Along with age, remission rates and overall survival (OS) depend on a number of other factors, including cytogenetics, previous bone marrow disorders (such as myelodysplastic syndromes [MDS]) and comorbidities. Currently, there is no effective cure for the disease. Therefore, there remains a current and urgent need for novel therapies directed toward the treatment and prevention of acute myeloid leukemia. Such therapies would desirably possess at least one of increased efficacy, lower number of side-effects, less severe side-effects, reduction of development of drug resistance, reduces the amount of drugs administered to a sub-optimal or sub-clinical dose as compared to individual drug administration, and/or the decrease of symptoms.

Gilteritinib has the chemical nomenclature 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide (hereinafter, may be referred to as “compound A”), and the following structure:

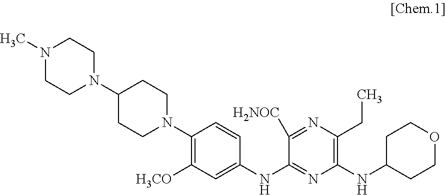 [SMILES: CCc1nc(C(N)=O)c(Nc2ccc(N3CCC(N4CCN(C)CC4)CC3)c(OC)c2)nc1NC1CCOCC1]

The compound A, or a salt thereof, is described in Patent Literature 1 (PTL 1) and Patent Literature 2 (PTL 2), and can be prepared in the same method of PTL 1. A preferred salt of the compound A is the hemifumarate salt. The compound A, or a salt thereof, is a FLT3 inhibitor under development for the treatment of AML (Future Oncol., 11(18), 2499-2501 (2015), 50th Annu. Meet. Am. Chem. Soc. Clin. Oncol. (ASCO) 2014, abst. 7070, 50th Annu. Meet. Am. Chem. Soc. Clin. Oncol. (ASCO) 2014, abst. 7071, 51^{th} Annu. Meet. Am. Chem. Soc. Clin. Oncol. (ASCO) 2015, abst. 7003). The compound A, or a salt thereof, also has inhibitory activities for AXL, leukocyte receptor tyrosine kinase (LTK), and anaplastic lymphoma kinase (ALK). The compound A, or a salt thereof, is preferably administered orally.

Azacitidine has the nomenclature 4-amino-1-β-D-ribofuranosyl-1,3,5-triazin-2(1H)-one, and 5-azacitidine has the following structure:

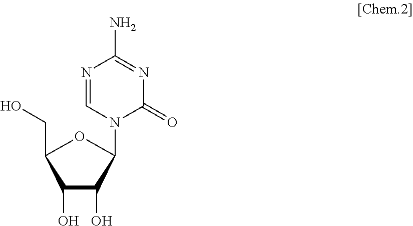

Azacitidine is an approved drug sold under the name Vidaza (registration symbol). Vidaza is a nucleoside metabolic inhibitor (hypomethylating agent) indicated for the treatment of patients with the following FAB myelodysplastic syndrome (MDS) subtypes: Refractory anemia (RA) or refractory anemia with ringed sideroblasts (RARS) (if accompanied by neutropenia or thrombocytopenia or requiring transfusions), refractory anemia with excess blasts (RAEB), refractory anemia with excess blasts in transformation (RAEB-T), and chronic myelomonocytic leukemia (CMMoL). The recommended starting dose for the first treatment cycle, for all patients regardless of baseline hematology values, is Vidaza 75 mg/m² daily for 7 days to be administered by subcutaneous (SC) injection or intravenous (IV) infusion. The patient may be pre-medicated for nausea and vomiting. The treatment cycles can be repeated every 4 weeks. After 2 cycles, the dose may be increased to 100 mg/m² if no beneficial effect is seen and no toxicity other than nausea and vomiting has occurred. Patients should be treated for a minimum of 4 to 6 cycles. Complete or partial response may require additional treatment cycles.

### CITATION LIST

### Patent Literature

PTL 1: WO2010/128659

PTL 2: WO2015/119122

### SUMMARY OF INVENTION

The present invention may address one or more of the above needs by providing alternative therapies for the treatment of AML, including those patients which present with the FLT3 mutation.

### BRIEF DESCRIPTION OF DRAWINGS

FIG. 1 shows Annexin-V-positive population in MV4-11 cells treated with compound A hemifumarate in combination with azacitidine. Mean with SE of 3 independent experiments. *** is P<0.001 compared with the value of the compound A hemifumarate-only treated group and ##is P<0.01, ###is P<0.001 compared with the value of the azacitidine-only treated group (Student's t-test). The vertical axis indicates Annexin-V-positive population in MV4-11 cells and the horizontal axis indicates compound A at final concentrations of 0 (DMSO), 1, 3 or 10 nmol/L in combination with azacitidine at final concentrations of 0 (DMSO) or 1000 nmol/L.

FIG. 2 shows Expression of proteins after treatment of MV4-11 cells with compound A hemifumarate with or without azacitidine.

FIG. 3 shows Antitumor effect of compound A hemifumarate in combination with azacitidine in nude mice xenografted with MV4-11 cells. *** is P<0.001 compared with the value of the compound A hemifumarate-treated group and +++is P<0.001 compared with the value of the azacitidine-treated group on Day 21 (Student's t-test).The vertical axis indicates Tumor volume (mm³) and the horizontal axis indicates the number of days.

### DESCRIPTION OF EMBODIMENTS

The present invention provides methods for treating acute myeloid leukemia which comprises administering to a patient in need thereof a therapeutically effective combination of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and 4-amino-1-β-D-ribofuranosyl-1,3,5-triazin-2(1H)-one, or a salt thereof. In an embodiment, the acute myeloid leukemia is an acute myeloid leukemia with FLT3 mutation. In an embodiment, the acute myeloid leukemia is mutant FLT3 polynucleotide-positive acute myeloid leukemia, FLT3 internal tandem duplication (ITD) positive acute myeloid leukemia, or acute myeloid leukemia with FLT3 point mutation. In an embodiment, the compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate. In an embodiment, 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, or hemifumarate thereof, is administered orally.

The present invention provides compositions for treating cancer, comprising 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, as an active ingredient, which is used for combined administration with 4-amino-1-β-D-ribofuranosyl-1,3,5-triazin-2(1H)-one (azacitidine), or a salt thereof. The present invention provides compositions for treating cancer, comprising 4-amino-1-β-D-ribofuranosyl-1,3,5-triazin-2(1H)-one, or a salt thereof, as an active ingredient, which is used for combined administration with 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof. In an embodiment of the compositions, the cancer is acute myeloid leukemia. In an embodiment of the compositions, the cancer is an acute myeloid leukemia with FLT3 mutation. In an embodiment of the compositions, the cancer is mutant FLT3 polynucleotide-positive acute myeloid leukemia, FLT3 internal tandem duplication (ITD) positive acute myeloid leukemia, or acute myeloid leukemia with FLT3 point mutation. In an embodiment of the compositions, the compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate.

The present invention provides uses of a therapeutically effective combination of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and 4-amino-1-β-D-ribofuranosyl-1,3,5-triazin-2(1H)-one, or a salt thereof, for treating acute myeloid leukemia in a patient in need thereof. In an embodiment, the acute myeloid leukemia is an acute myeloid leukemia with FLT3 mutation. In an embodiment, the acute myeloid leukemia is mutant FLT3 polynucleotide-positive acute myeloid leukemia, FLT3 internal tandem duplication (ITD) positive acute myeloid leukemia, or acute myeloid leukemia with FLT3 point mutation. In an embodiment, the compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate. In an embodiment, 6-ethyl-β-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, or hemifumarate thereof, is administered orally.

The present invention provides uses of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, in the manufacture of a medicament for treating acute myeloid leukemia in combination with 4-amino-1-β-D-ribofuranosyl-1,3,5-triazin-2(1H)-one, or a salt thereof. The present invention provides uses of 4-amino-1-β-D-ribofuranosyl-1,3,5-triazin-2(1H)-one, or a salt thereof, in the manufacture of a medicament for treating acute myeloid leukemia in combination with 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof. In an embodiment of the uses, the acute myeloid leukemia is an acute myeloid leukemia with FLT3 mutation. In an embodiment of the uses, the acute myeloid leukemia is mutant FLT3 polynucleotide-positive acute myeloid leukemia, FLT3 internal tandem duplication (ITD) positive acute myeloid leukemia, or acute myeloid leukemia with FLT3 point mutation. In an embodiment of the uses, the compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate. In an embodiment of the uses, the medicament comprising said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, or hemifumarate thereof, is an oral medicament.

The present invention also provides

(1) A method for treating acute myeloid leukemia which comprises administering to a patient in need thereof a therapeutically effective combination of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and 4-amino-1-β-D-ribofuranosyl-1,3,5-triazin-2(1H)-one, or a salt thereof.

(2) The method according to (1), wherein said acute myeloid leukemia is an acute myeloid leukemia with FLT3 mutation.

(3) The method according to (1) or (2), wherein said acute myeloid leukemia is mutant FLT3 polynucleotide-positive acute myeloid leukemia, FLT3 internal tandem duplication (ITD) positive acute myeloid leukemia, or acute myeloid leukemia with FLT3 point mutation.

(4) The method according to any of (1)-(3), wherein said compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate.

(5) The method according to any of (1)-(4), wherein said 6-ethyl-3-({3-methoxy-4-[4-(4-methylpiperazin-1-yl)piperidin-1-yl]phenyl}amino)-5-( tetrahydro-2H-pyran-4-ylamino)pyrazine-2-carboxamide, or a salt thereof, or hemifumarate thereof, is administered orally.

(6) The method according to any of (1)-(5), wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, or hemifumarate thereof, and said 4-amino-1-β-D-ribofuranosyl-1,3,5-triazin-2(1H)-one, or a salt thereof, are administered simultaneously.

(7) The method according to any of (1)-(5), wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, or hemifumarate thereof, and said 4-amino-1-β-D-ribofuranosyl-1,3,5-triazin-2(1H)-one, or a salt thereof, are administered sequentially.

(8) The method according to any of (1)-(5), wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, or hemifumarate thereof, and said 4-amino-1-β-D-ribofuranosyl-1,3,5-triazin-2(1H)-one, or a salt thereof, are administered in a single unit dose.

(9) The method according to any of (1)-(5), wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, or hemifumarate thereof, and said 4-amino-1-β-D-ribofuranosyl-1,3,5-triazin-2(1H)-one, or a salt thereof, are administered in separate dosage forms.

(10) The method according to (1), wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, is administered in a dosage of about 0.001 mg/kg patient weight to about 100 mg/kg patient weight.

(11) The method according to (10), wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, is administered orally.

(12) The method according to (1), wherein said 4-amino-1-β-D-ribofuranosyl-1,3,5-triazin-2(1H)-one, or a salt thereof, is administered in a dosage of about 5 mg/m² patient surface area to about 125 mg/m² patient surface area.

(13) The method according to any of (1)-(9), wherein said 4-amino-1-β-D-ribofuranosyl-1,3,5-triazin-2(1H)-one, or a salt thereof, is administered via subcutaneous injection or intravenous infusion.

(14) A composition for treating cancer, comprising 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, in combination with 4-amino-1-β-D-ribofuranosyl-1,3,5-triazin-2(1H)-one (azacitidine), or a salt thereof.

(15) The composition of (14), wherein said compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate.

(16) Use of a therapeutically effective combination of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and 4-amino-1-β-D-ribofuranosyl-1,3,5-triazin-2(1H)-one, or a salt thereof, for treating acute myeloid leukemia in a patient in need thereof.

(17) The use of (16), wherein said acute myeloid leukemia is an acute myeloid leukemia with FLT3 mutation.

(18) The use of (16), wherein said acute myeloid leukemia is mutant FLT3 polynucleotide-positive acute myeloid leukemia, FLT3 internal tandem duplication (ITD) positive acute myeloid leukemia, or acute myeloid leukemia with FLT3 point mutation.

(19) The use according to any of (16)-(18), wherein said compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate.

(20) The use according to any of (16)-(18), wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, is administered orally.

The present invention also provides

(1) A method for treating acute myeloid leukemia which comprises administering to a patient in need thereof a therapeutically effective combination of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and 4-amino-1-β-D-ribofuranosyl-1,3,5-triazin-2(1H)-one, or a salt thereof.

(2) The method according to (1), wherein said acute myeloid leukemia is an acute myeloid leukemia with FLT3 mutation.

(3) The method according to (1) or (2), wherein said acute myeloid leukemia is mutant FLT3 polynucleotide-positive acute myeloid leukemia, FLT3 internal tandem duplication (ITD) positive acute myeloid leukemia, or acute myeloid leukemia with FLT3 point mutation.

(4) The method according to any of (1)-(3), wherein said compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate.

(5) The method according to any of (1)-(4), wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, is administered orally.

(6) A composition for treating cancer, comprising 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, as an active ingredient, which is used for combined administration with 4-amino-1-β-D-ribofuranosyl-1,3,5-triazin-2(1H)-one (azacitidine), or a salt thereof.

(7) A composition for treating cancer, comprising 4-amino-1-β-D-ribofuranosyl-1,3,5-triazin-2(1H)-one, or a salt thereof, as an active ingredient, which is used for combined administration with 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof.

(8) The composition of (6) or (7), wherein said cancer is acute myeloid leukemia.

(9) The composition of any of (6)-(8), wherein said cancer is an acute myeloid leukemia with FLT3 mutation.

(10) The composition of any of (6)-(9), wherein said cancer is mutant FLT3 polynucleotide-positive acute myeloid leukemia, FLT3 internal tandem duplication (ITD) positive acute myeloid leukemia, or acute myeloid leukemia with FLT3 point mutation.

(11) The composition of any of (6)-(10), wherein said compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate (HFM).

(12) The use of a therapeutically effective combination of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and 4-amino-1-β-D-ribofuranosyl-1,3,5-triazin-2(1H)-one, or a salt thereof, for treating acute myeloid leukemia in a patient in need thereof.

(13) The use of (12), wherein said acute myeloid leukemia is an acute myeloid leukemia with FLT3 mutation.

(14) The use of (12) or (13), wherein said acute myeloid leukemia is mutant FLT3 polynucleotide-positive acute myeloid leukemia, FLT3 internal tandem duplication (ITD) positive acute myeloid leukemia, or acute myeloid leukemia with FLT3 point mutation.

(15) The use according to any of (12)-(14), wherein said compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate (HFM).

(16) The use according to any of (12)-(15), wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, is administered orally.

(17) Use of 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, in the manufacture of a medicament for treating acute myeloid leukemia in combination with 4-amino-1-β-D-ribofuranosyl-1,3,5-triazin-2(1H)-one, or a salt thereof.

(18) Use of 4-amino-1-β-D-ribofuranosyl-1,3,5-triazin-2(1H)-one, or a salt thereof, in the manufacture of a medicament for treating acute myeloid leukemia in combination with 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof.

(19) The use of (17) or (18), wherein said acute myeloid leukemia is an acute myeloid leukemia with FLT3 mutation.

(20) The use of any of (17)-(19), wherein said acute myeloid leukemia is mutant FLT3 polynucleotide-positive acute myeloid leukemia, FLT3 internal tandem duplication (ITD) positive acute myeloid leukemia, or acute myeloid leukemia with FLT3 point mutation.

(21) The use according to any of (17)-(20), wherein said compound is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate(HFM).

(22) The use according to any of (17) or (19)-(21), wherein said medicament comprising said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, is an oral medicament.

Acute myeloid leukemia includes acute myeloid leukemias with FLT3 mutation. An acute myeloid leukemia with FLT3 mutation includes mutant FLT3 polynucleotide-positive acute myeloid leukemia, FLT3 internal tandem duplication (ITD) positive acute myeloid leukemia, or acute myeloid leukemia with FLT3 point mutation.

FLT3 is a member of the class III receptor tyrosine kinase (TK) family that is normally expressed on the surface of hematopoietic progenitor cells. FLT3 and its ligand play an important role in proliferation, survival and differentiation of multipotent stem cells. FLT3 is overexpressed in the majority of AML cases. In addition, activated FLT3 with internal tandem duplication (ITD) in and around the juxtamembrane domain and tyrosine kinase domain (TKD) mutations at around D835 in the activation loop are present in 28% to 34% and 11% to 14% of AML cases, respectively. These activated mutations in FLT3 are oncogenic and show transforming activity in cells. Patients with FLT3-ITD mutation show poor prognosis in clinical studies, with a higher relapse rate, a shorter duration of remission from initial therapy (6 months versus 11.5 months for those without FLT3-ITD mutations) as well as reduced disease-free survival (16% to 27% versus 41% at 5 years) and OS (15% to 31% versus 42% at 5 years). The incidence of relapse after hematopoietic stem cell transplant (HSCT) is also higher for patients with FLT3-ITD (30% versus 16% at 2 years for those without FLT3-ITD mutations). Similar to their prognosis for first line therapy, patients with relapsed/refractory FLT3-mutation positive AML have lower remission rates with salvage chemotherapy, shorter durations of remission to second relapse and decreased OS relative to FLT3-mutation negative patients.

AXL tyrosine kinase (AXL) is a member of TAM family (Tyro-3, AXL and Mer) receptor TKs and is normally expressed in cells of mesenchymal origin, such as osteoblasts, fibroblasts and blood cells. AXL has been reported to be overexpressed or activated in many cancers, including AML. AXL overexpression in AML confers drug resistance and is associated with adverse prognosis. AXL inhibition suppresses the growth of human FLT3-positive AML in vivo. In addition, AXL inhibition is also effective against FLT3-negative AML expressing AXL in vivo.

The salts of compound A and azacitidine may be used in the present invention. “Salts” refers to pharmaceutically acceptable derivatives of the disclosed compounds wherein the parent compound is modified by converting an existing acid or base moiety to its salt form. Lists of suitable salts are found in Remington's Pharma- ceutical Sciences, 17th ed., Mack Publishing Company, Easton, Pa., 1985, p. 1418 and Journal of Pharmaceutical Science, 66, 2 (1977).

The terms “treating”, “treat”, “to treat”, or “treatment” include restraining, slowing, stopping, reducing, or reversing the progression or severity of an existing symptom, disorder, condition, or disease.

The term “patient” refers to a mammal, preferably a human.

The term “about” generally indicates a possible variation of no more than 10%, 5%, or 1% of a value. For example, “about 25 mg/kg” will generally indicate, in its broadest sense, a value of 22.5-27.5 mg/kg, i.e., 25±2.5 mg/kg.

“Therapeutically effective amount” is an amount of the compounds when ad- ministered in combination to a patient treats acute myeloid leukemia. An amount that proves to be a therapeutically effective amount in a given instance, for a particular subject, may not be effective for 100% of subjects similarly treated for the disease, even though such dosage is deemed a therapeutically effective amount by skilled practitioners. The amount of the compound that corresponds to a therapeutically effective amount is strongly dependent on the particular type of cancer, stage of the cancer, the age of the patient being treated, and other factors. In general, therapeutically effective amounts of these compounds are well-known in the art. In addition, a therapeutically effective amount may be a combination amount where one or both of compound A, or a salt thereof, and azacitidine are administered in a sub-therapeutically effective amount or dosage, but results in treating the acute myeloid leukemia. A subtherapeutically effective amount is an amount of a compound that, when administered to a patient by itself, does not completely inhibit over time the biological activity of the intended target.

The present invention includes the use or administration of the combination in a therapeutically effective interval. “Therapeutically effective interval” is a period of time beginning when one of the compounds is administered to a patient and ending at the limit of the administration of the other compound where the benefit of the combination administration of the two compounds is retained. The combination administration, therefore, can be simultaneous or sequential, and in any order.

The time period or cycle for the combination administration can be for a total of one week, 28 days, one, two, three, or four months, or more. The individual drugs can each be administered every day for the entire duration of the period or cycle, or only a portion thereof. For instance, on a 28 day cycle, compound A, or a salt thereof, can be administered every day in the cycle while azacitidine can be administered for just a portion thereof, such as for 5 consecutive days, 7 consecutive days, or 10 consecutive days, and the 5, 7, and 10 consecutive days can be the first 5, 7, or 10 days of the period or cycle, respectively.

The compounds, or their salts, can be administered via any of the accepted modes of administration or agents known in the art. The compounds may be administered, for example, orally, nasally, parenterally (intravenous, intramuscular, or subcutaneous), topically, transdermally, intravaginally, intravesically, intracistemally, or rectally. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, preferably in unit dosage forms suitable for simple administration of precise dosages. A preferred route of administration for compound A, or a salt thereof, is oral, while preferred routes of administration of azacitidine are subcutaneous and infusion.

The compounds can be administered in a single unit dose or separate dosage forms, and the formulations used in the forms can include other active ingredients and/or known carriers. Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like. How to prepare the formulations and forms are known to those of ordinary skill in the art, and examples are provided, for instance, in Remington's Pharmaceutical Sciences, 18th Ed. (Mack Publishing Company, Easton, Pa., 1990).

The amounts of the two compounds which are administered to a patient can be determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific neoplasm involved; the degree of or involvement or the severity of the neoplasm; the response of the in- dividual patient; the particular compound administered; the mode of administration; the bioavailability characteristics of the preparation administered; the dose regimen selected; the use of concomitant medication; and other relevant circumstances. For example, when compound A, or a salt thereof, is orally administered, the daily dose can be from about 0.001 to about 100 mg/kg, preferably about 0.005 to about 30 mg/kg, more preferably suitably about 0.01 to about 10 mg/kg, per body mass of the patient. In some embodiments, compound A, or a salt thereof, is administered in an amount of about 80 mg per day. When administered intravenously, the daily dose can be suitably from about 0.0001 to about 10 mg/kg per body mass of the patient, the total administered by dividing into one or more doses in a day. In addition, a transmucosal agent is administered at a dose from about 0.001 to about 100 mg/kg per body weight, and can be administered once a day or divided and administered several times in a day. Azacitidine can be administered in an amount from about 5 mg/m² to about 125 mg/m², from about 50 mg/m² to about 100 mg/m²and more preferably in an amount of about 75 mg/m₂, surface area of the patient. Azacitidine can be administered in an amount of about 250 to about 500 mg per day.

### Example 1

Induced Apoptosis in MV4-11 Cells

MV4-11, a cell line derived from human AML and which harbors the FLT-3-ITD mutation, was purchased from American Type Culture Collection (ATCC). The cells were cultured at 37 ° C. in 5% CO₂ in Iscove's Modified Dulbecco's Medium supplemented with 10% heat-inactivated fetal bovine serum. The cells were seeded on 12 well plates and were cultured overnight. The cells were treated with compound A at final concentrations of 0 (DMSO), 1, 3 or 10 nmol/L in combination with azacitidine (Tokyo Chemical Industry) at final concentrations of 0 (DMSO) or 1000 nmol/L. After forty-eight hours, cells were harvested and incubated with Guava (registration symbol) Nexin Reagent (Merck Millipore), and annexin-V-positive cells were determined using a Guava (registration symbol) PCA microcytometer (Guava Technologies). The percentage of annexin-V-positive cells in each sample was analyzed using CytoSoft software (Guava Technologies). Mean and standard error (SE) values were obtained from three independent experiments.

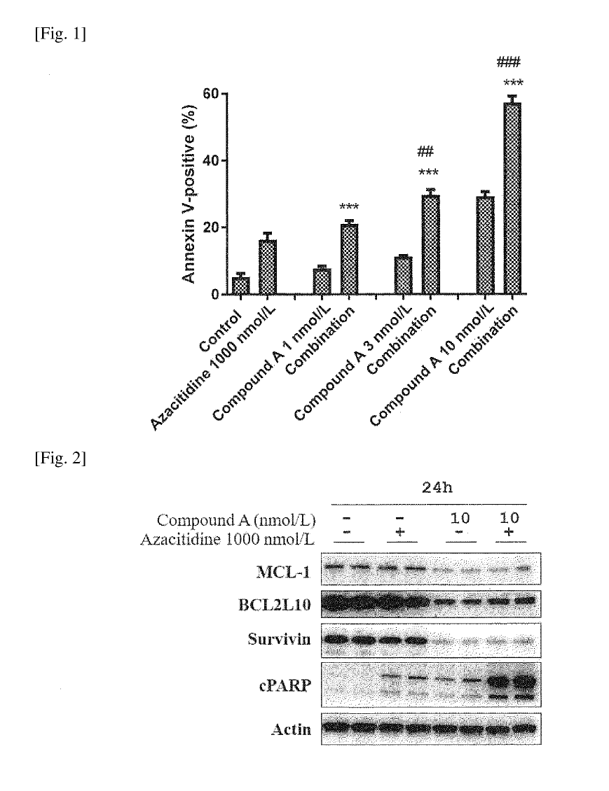

The treatment with compound A at concentrations of 3 and 10 nmol/L, in combination with azacitidine at a concentration of 1000 nmol/L, for 48 hours significantly increases the annexin-V-positive population in MV4-11 cells, as compared to those in either the compound A hemifumarate-only or the azacitidine-only treated cells (using Student's t-test) (FIG. 1).

The results indicate that compound A at concentrations of 3 and 10 nmol/L, in combination with azacitidine at 1000 nmol/L, induces apoptosis in the MV4-11 cells.

### Example 2

Anti-Apoptosis Protein Expression

MV4-11 cells were seeded on 15 cm dish and cultured overnight. The cells were treated with compound A at 0 (DMSO) or 10 nmol/L in combination with azacitidine at 0 (DMSO) or 1000 nmol/L at final concentration. The assay was performed in duplicate. After twenty-four hours, cells were harvested and lysed with lysis buffer (RIPA Buffer [Thermo Fisher Scientific], 1xHalt Phosphatase Inhibitor Cocktail [Thermo Fisher Scientific], and Protease Inhibitor Cocktail [Sigma-Aldrich]). The samples are centrifuged, and protein concentrations of supernatants were determined using the Pierce (trademark) 660nm Protein Assay (Thermo Fisher Scientific). Aliquots of 2.0 μg protein/μL, were prepared in sample buffer (10 mmol/L dithiothreitol (DTT) [Nacalai Tesque] and 1xSDS sample buffer [Wako Pure Chemical Industries, Ltd.], in lysis buffer), and then were boiled for 5 min.

The samples were separated by electrophoresis and transferred with Trans-Blot (registration symbol) Turbo transfer Pack PVDF (Bio-Rad Laboratories). After blocking with Blocking One (Nacalai Tesque) for 1 hour, each membrane was incubated with antibodies against MCL-1 (#5453, Cell Signaling Technology), BCL2L10 (#3869, Cell Signaling Technology), Survivin (#AF886, R&D Systems), cleaved PARP (#9541, Cell Signaling Technology), or Actin (A2066, Sigma-Aldrich) overnight in a cold room. After washing, the membranes were then incubated with an anti-rabbit IgG HRP-linked antibody (#7074, Cell Signaling Technology) for 1 hour at room temperature. After a final wash, signals for the each proteins were detected using a chemiluminescence reagent ECL-prime Blotting Detection Reagent (GE Healthcare) with a CCD camera (ImageQuant LAS4000, GE Healthcare).

Compound A hemifumarate inhibits the expression in the MV4-11 cells of anti-apoptosis proteins MCL-1, BCL2L10 and survivin. (See FIG. 2). An increase in PARP cleavage was also observed in MV4-11 cells following compound A hemifumarate treatment; cells co-treated with compound A hemifumarate and azacitidine showed a further increase in PARP cleavage.

### Example 3

MV4-11 Xenografted Mouse Model

Four-week-old male nude mice {CAnN.Cg-Foxnlnu/CrlCrlj(nu/nu)}were purchased from Charles River Laboratories Japan, Inc. MV4-11 cells were subcutaneously inoculated into the flank at 5×10⁶ cells/0.1 mL/mouse and allowed to grow. Mice with tumor volumes (length x width²×0.5) of 100 to 300 mm³ were selected one day before administration and divided into 4 groups (n=10), so that the mean tumor volume in each group was almost equal. Control group received once-daily oral administration of 0.5% methylcellulose solution from Days 0 to 20, and once-daily intravenous administration of saline from Days 0 to 4. The compound A hemifumarate group received once-daily oral administration of compound A hemifumarate at 3 mg/kg/day from Days 0 to 20, and once-daily intravenous administration of saline from Days 0 to 4. The azacitidine group received once-daily oral administration of 0.5% methyl-cellulose from Days 0 to 20, and once-daily intravenous administration of azacitidine at 3 mg/kg/day from Days 0 to 4. The combination group received once-daily oral administration of compound A hemifumarate at 3 mg/kg/day from Days 0 to 20, and once-daily intravenous administration of azacitidine at 3 mg/kg/day from Days 0 to 4.

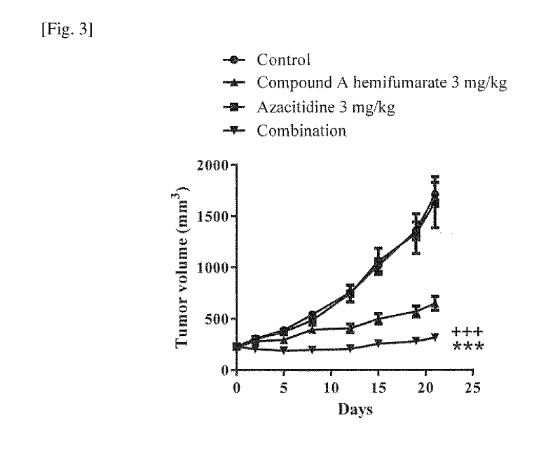

The result is shown on FIG. 3. The mean tumor volume on Day 21 in the combination group was significantly smaller compared to that in either the compound A hemifumarate-only treated or the azacitidine-only treated group (using Student's t-test).

In summary, compound A hemifumarate in combination with azacitidine shows superior antitumor efficacy in mice xenografted with MV4-11 cells compared to that of compound A hemifumarate-only or azacitidine-only treated groups.

### Example 4

Approximately 528 human subjects with newly diagnosed acute myeloid leukemia who are not eligible for intensive induction therapy are randomized in a 1:1:1 ratio to receive compound A hemifumarate (Arm A), compound A hemifumarate plus azacitidine (Arm AC), or azacitidine only (Arm C). The randomization is stratified based on age group described below:

1) Age75 years

2) Age <75 years

The treatment period is a 28-day cycle. For Arm A, the compound A hemifumarate starting oral dose is 120 mg/day. For Arm AC, the compound A hemifumarate starting oral dose is either 120 mg or 80 mg per day. For Arms AC and C, azacitidine is dosed at 75 mg/m² daily, for days 1-7 of the cycle, via subcutaneous injection or intravenous infusion. Dose increases and reductions are permitted for compound A hemifumarate and azacitidine. For example, the dose of compound A hemifumarate may be increased to 200 mg/day.

The subjects are monitored for the following parameters: overall survival (OS), event-free survival (EFS), complete remission (CR) rate, leukemia-free survival (LFS), duration of remission, composite complete remission (CRc) rate, patient reported fatigue (Brief Fatigue Inventory [BFI]), adverse events (AEs), transplantation rate, minimal residual disease (MRD), FLT3 gene mutation status (mutation types and frequency, relationship to efficacy and safety, mechanisms of acquired resistance), patient reported dyspnea (Functional Assessment of Chronic Illness Therapy-Dyspnea-Short Form [FACIT-Dys-SF]), patient reported signs, symptoms, and impacts of AML (Functional Assessment of Cancer Therapy-Leukemia [FACT-Leu] and dizziness and mouth sores items), health-related quality of life assessed by the EuroQol Group 5-dimension 5-level (EQ-5D-5L) instrument, and resource utilization including hospitalization, blood transfusion, antibiotic intravenous infusions, medication for AEs and opioid usage.

The subjects have an end-of-treatment visit within 7 days after treatment cessation, followed by a 30-day follow up for safety. After this, the subjects enter the long-term follow-up period for collection of subsequent treatment, remission status, EQ-5D-5L, and survival (cause and date of death).

### INDUSTRIAL APPLICABILITY

Utility of the combination therapy is illustrated by the positive impact had in one or more of the studies above, including on one or more of the parameters delineated.

1. A method for treating acute myeloid leukemia which comprises administering to a patient in need thereof a therapeutically effective combination of 6-ethyl-3-(3-methoxy-4-[4-(4-methylpiperazin-1-yl)piperidin-1-yl]phenyll amino)-5-(tetrahydro-2H-pyran-4-ylamino)pyrazine-2-carboxamide, or a salt thereof, and 4-amino-1-β-D-riboffiranosyl-1,3,5-triazin-2(1,H)-one, or a salt thereof.

2. The method according to claim 1, wherein said acute myeloid leukemia is an acute myeloid leukemia with FLT3 mutation.

3. The method according to claim 2, wherein said acute myeloid leukemia with FLT3 mutation is mutant FLT3 polynucleotide-positive acute myeloid leukemia, FLT3 internal tandem duplication (ITD) positive acute myeloid leukemia, or acute myeloid leukemia with FLT3 point mutation.

4. The method according to claim 1, wherein said 6 ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide or a salt thereof is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate.

5. The method according to claim 4, wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamicle hemifumarate is administered orally.

6. The method according to claim 1, wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, or hemifumarate thereof, and said 4-amino-1-β-D-ribofuranosyl-1,3,5-triazin-2(1H)-one, or a salt thereof, are administered simultaneously.

7. The method according to claim 1, wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and said 4-amino-1-β-D-ribofuranosyl-1,3,5-triazin-2(1H)-one, or a salt thereof, are administered sequentially.

8. The method according to claim 1, wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and said 4-amino-1-β-D-ribofuranosyl-1,3,5-triazin-2(H)-one, or a salt thereof, are administered in a single unit dose.

9. The method according to claim 1, wherein said 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide, or a salt thereof, and said 4-amino-1-β-D-ribofuranosyl-1,3,5-triazin-2(1H)-one, or a salt thereof, are administered in separate dosage forms.

10. The method according to claim 1, wherein said 6-ethyl-3-({3-methoxy-4[4-(4-methylpiperazin-1-yl)piperidin-1-yl]phenyl}amino)-5-(tetrahydro-2H-pyran-4-ylamino)pyrazine-2-carboxamide, or a salt thereof, is administered in a dosage of about 0.001 mg/kg patient weight to about 100 mg/kg patient weight.

11. The method according to claim 10, wherein said 6-ethyl-3-({3-metboxy-4[4-(4-methylpiperazin-1-yppiperidin-1-yl]phenyl}amino)-5-(tetrahydro-2H-pyran-4-ylamino)pyrazine-2-carboxamide, or a salt thereof, is administered orally.

12. The method according to claim 1, wherein said 4-amino-1-β-D-ribofuranosyl-1,3,5-triazin-2(1H)-one, or a salt thereof, is administered in a dosage of about 5 mg/m² patient surface area to about 125 mg/m² patient surface area.

13. The method according to claim 9, wherein said 4-amino-1-β-D-ribofuranosyl-1,3,5-triazin-2(1H)-one, or a salt thereof, is administered via subcutaneous injection or intravenous infusion.

14. A composition, comprising 6-ethyl-3-({3-methoxy-4-[4-(4-methylpiperazin-1-yl)piperidin-1-yl]phenyl}(amino)-5-(tetrahydro-2H-pyran-4-ylamino)pyrazine-2-carboxamide, or a salt thereof, in combination with 4-amino-1-β-D-ribofurartosyl-1,3,5-triazin-2(1H)-one (azacitidine), or a salt thereof.

15. The composition of claim 14, wherein said 6ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide or a salt thereof is 6-ethyl-3-({3-methoxy-4-[4-(4-methylpiperazin-1-yl)piperidin-1-yl]phenyl}amino)-5-(tetrahydro-2H-pyran-4-ylamino)pyrazine-2-carboxamide hemifumarate.

16-20. (canceled)
